(19) United States
(12) Patent Application Publication
(10) Pub. No.: US 2011/0022074 A1
(43) Pub. Date: Jan. 27, 2011

(54) PERMANENT ARTERIAL EMBOLI DISSOLUTION FILTER TO PREVENT EMBOLIC OCCLUSION OF A BLOOD VESSEL

(76) Inventor: Alex Powell, Miami, FL (US)

Correspondence Address: GOLD & RIZVI, P.A., 11575 Heron Bay Blvd., SUITE 309, Coral Springs, FL, 33076, US

(21) Appl. No.: 12/840,881

(22) Filed: Jul. 21, 2010

Related U.S. Application Data
(60) Provisional application No. 61/213,856, filed on Jul. 22, 2009.

(51) Int. Cl. A61F 2/01

(52) U.S. Cl. 606/200

(57) ABSTRACT

An arterial emboli dissolution filter apparatus is a permanently implanted for the prevention of embolic occlusion of a blood vessel. The dissolution filter comprises an anchor section, wherein the anchor section is similar to a stent. The dissolution filter is attached to the anchor section. The device is designed so that normal blood can flow freely though the filter element while any potential embolism will be retained by the filtering element. The passing blood flow is used to dissolve and dissolve any retained emboli. The dissolution filter is fabricated of any reasonably arranged lattice elements forming a series of mesh pores. The filter material and design directs the emboli and blood flow towards a central location, thus increasing the efficiency in dissolving the emboli.

### RELATED US PATENT APPLICATION

This Non-Provisional US Application claims priority to Provisional U.S. Application 61/213,856, filed Jul. 22, 2009, which is incorporated in its entirety by reference herein.

### FIELD OF THE INVENTION

The present disclosure generally relates to an apparatus and method for the prevention of possible embolization by placement of an arterial diffuser. In particular this invention relates to the implantation of a permanent arterial filtering device that traps any potential embolism.

### BACKGROUND OF THE INVENTION

Embolic stroke is one of the leading causes of stroke today. It is estimated that up to 25% of strokes are embolic in etiology. Typically patients with embolic stroke have Atrial Fibrillation although other conditions that lead to thrombus formation and subsequent embolization exist. In addition to preventing an embolic stroke, this device may be placed in multiple locations throughout the arterial system to prevent embolic occlusion at any desired location.

Blood to the brain hemispheres is supplied by two carotid arteries, each of which branches-off into a so-called internal carotid and an external carotid. Blood is supplied to the brain stem by two vertebral arteries.

Cerebralvascular diseases are considered among the leading causes of mortality and morbidity in the modern age. Strokes denote an abrupt impairment of brain function caused by pathologic changes occurring in blood vessels. The main cause of strokes is insufficient blood flow to the brain (referred to as “an ischemic stroke”), which are about 80% of stroke cases.

Ischemic strokes are caused by sudden occlusion of an artery supplying blood to the brain. Occlusion or partial occlusion (stenosis) is the result of diseases of the arterial wall. Arterial atherosclerosis is by far the most common arterial disorder, and when complicated by thrombosis or embolism it is the most frequent cause of cerebral ischemia and infarction, eventually causing the cerebral stroke.

Such disorders are treated in different ways such as by drug management, surgery (carotid endarterectomy) in case of occlusive disease, or carotid angioplasty and carotid stents as known in the art.

While endarterectomy, angioplasty, and carotid stenting are procedures targeting at reopening the occluded artery, they do not prevent progression of new plaque (restenosis). Furthermore, embolisms from the new forming plaque in the internal carotid artery (with or without a stent implanted therein) can occlude smaller arteries in the brain and cause strokes. Even more so, the above treatment methods do not prevent proximal embolic sources, i.e. embolus formed at remote sites (heart and ascending aorta) to pass through the reopened stenosis in the carotid and occlude smaller arteries in the brain.

It will also be appreciated that endarterectomy is not suitable for intracranial arteries or in the vertebrobasilar system since these arteries are positioned within unacceptable environment (brain tissue, bone issue) or are of a small diameter.

Medically known as a cerebrovascular accident (CVA), a stroke is the rapidly developing loss of brain function(s) due to disturbance in the blood supply to the brain. This can be due to ischemia (lack of blood flow) caused by blockage (thrombosis, arterial embolism), or a hemorrhage (leakage of blood). As a result, the affected area of the brain is unable to function, leading to inability to move one or more limbs on one side of the body, inability to understand or formulate speech, or inability to see one side of the visual field.

A stroke is a medical emergency and can cause permanent neurological damage, complications, and even death. It is the leading cause of adult disability in the United States and Europe and it is the number two cause of death worldwide. Risk factors for stroke include advanced age, hypertension (high blood pressure), previous stroke or transient ischemic attack (TIA), diabetes, high cholesterol, cigarette smoking and atrial fibrillation. High blood pressure is the most important modifiable risk factor of stroke.

A stroke is occasionally treated with thrombolysis (“clot buster”), in the “stroke unit” of a hospital. Secondary prevention may involve antiplatelet drugs (aspirin and often dipyridamole), blood pressure control, statins, and in selected patients with carotid endarterectomy and anticoagulation. Treatment to recover lost function is stroke rehabilitation, involving health professions such as speech and language therapy, physical therapy and occupational therapy.

Another treatment for preventing strokes is provided by implanting filtering or trapping means into blood vessels. Introducing filtering means into blood vessels has been known for a while, in particular into veins. However, such filtering means are generally of a complex design, which render such devices not suitable for implantation with carotid arteries and not suitable for handling fine plaque debris. However, when considering the possible cerebral effects of even fine plaque debris occluding an artery supplying blood to the brain, the consequences may be fatal or cause irreversible brain damage.

Whilst a large variety of patents in the field of implantable filtering systems are known, they are mostly intended for implantation in veins and in particular are intended for vena cava implantation. An inferior vena cava filter, also IVC filter a type of vascular filter, is a medical device that is implanted into the inferior vena cava to prevent fatal pulmonary emboli (PEs).

An exemplary blood filtering apparatus is designed to collect plaque debris. The filter is implanted into the patient's blood vessel and monitored to determine a change in pressure or flow. The trap element is designed for trapping plaque debris which enter the filtering unit, and which owing to the essentially unidirectional blood flow, are drifted into the trap element where thy are entrapped by the trapping members, preventing the plaque debris from flowing upstream to the filtering unit.

The trap element is provided for trapping plaque debris, which are screened at the filtering unit but after a while might have passed through the openings of the filtering unit. The trapping elements are flexible to entrap and remove the plaque debris from the blood flow. The unit directs the blood flow away from the collection portion of the implanted trap. One such means is the inclusion of horseshoe shaped apertures with their leg portions extending upstream. The center portion projects slightly outward directing the flow about the filtering device.

The major drawback of these devices is the requirement for replacement when the filtering unit becomes blocked. The diversity of the flow velocity profile gives indication as to the degree of occlusion of the trap element enabling professional staff to determine when it is necessary to remove the plaque debris entrapped within the trap element. This dictates follow up surgical procedures, which is undesirable for the patient and degrades the vessels.

Therefore, it would be desirable to have a permanently implantable dissolution device that is positioned in a blood vessel supplying blood to the brain so as to filter the blood and utilize the blood flow to dissolve clots and thereby preventing an embolic stroke.

### SUMMARY OF THE INVENTION

The present disclosure is generally directed to an apparatus and respective method for trapping and dispersing potential emboli, the method comprising the steps of:

obtaining a emboli dissolution device, the emboli dissolution device comprising:
a filter support, and
a dissolution filter comprising a filter mesh, the dissolution filter designed to retain emboli and direct blood flow towards the retained clots to dissolve the clots;

permanently implanting the emboli dissolution device within a blood vessel of a patient; and

dispersing an emboli by retaining the emboli using the dissolution filter and directing blood flow towards the retained emboli.

In a second aspect, the emboli dissolution method further comprises the step of supporting the dissolution filter by positioning the filter support against an interior surface of the blood vessel.

In another aspect, the method further comprises the step of providing unimpeded flow through the dissolution filter.

In another aspect, the method further comprises the step of concentrating the blood flow towards a central portion of the dissolution filter.

In another aspect, the method further comprises a step of retaining smaller particles proximate a central portion of the dissolution filter, wherein the central portion of the dissolution filter comprises a finer mesh than an outer portion of the dissolution filter.

In another aspect, the dissolution filter is fabricated having a mesh comprising a series of spatially arranged longitudinal elements.

In another aspect, the dissolution filter is fabricated having a mesh comprising a series of spatially arranged lateral elements.

In another aspect, the dissolution filter is fabricated having a mesh comprising a series of spatially arranged radial elements.

These and other features, aspects, and advantages of the invention will be further understood and appreciated by those skilled in the art by reference to the following written specification, claims and appended drawings.

### BRIEF DESCRIPTION OF THE DRAWINGS

The invention will now be described, by way of example, with reference to the accompanying drawings, where like numerals denote like elements and in which:

FIG. 1 presents an exemplary sectioned view of a vessel wall illustrating an installation of a emboli dissolution filter apparatus;

FIG. 2 presents an enlarged view of the exemplary emboli dissolution filter apparatus introduced in FIG. 1;

FIG. 3 presents a top plan view of the exemplary emboli dissolution filter apparatus introduced in FIG. 1;

FIG. 4 presents an exemplary sectioned view of the vessel wall illustrating an installation of a first alternate emboli dissolution filter apparatus;

FIG. 5 presents an enlarged view of the first alternate exemplary emboli dissolution filter apparatus introduced in FIG. 4;

FIG. 6 presents a top plan view of the first alternate exemplary emboli dissolution filter apparatus introduced in FIG. 4; and

FIG. 7 presents a top plan view of a second alternate exemplary emboli dissolution filter apparatus.

Like reference numerals refer to like parts throughout the various views of the drawings.

### DETAILED DESCRIPTION OF THE INVENTION

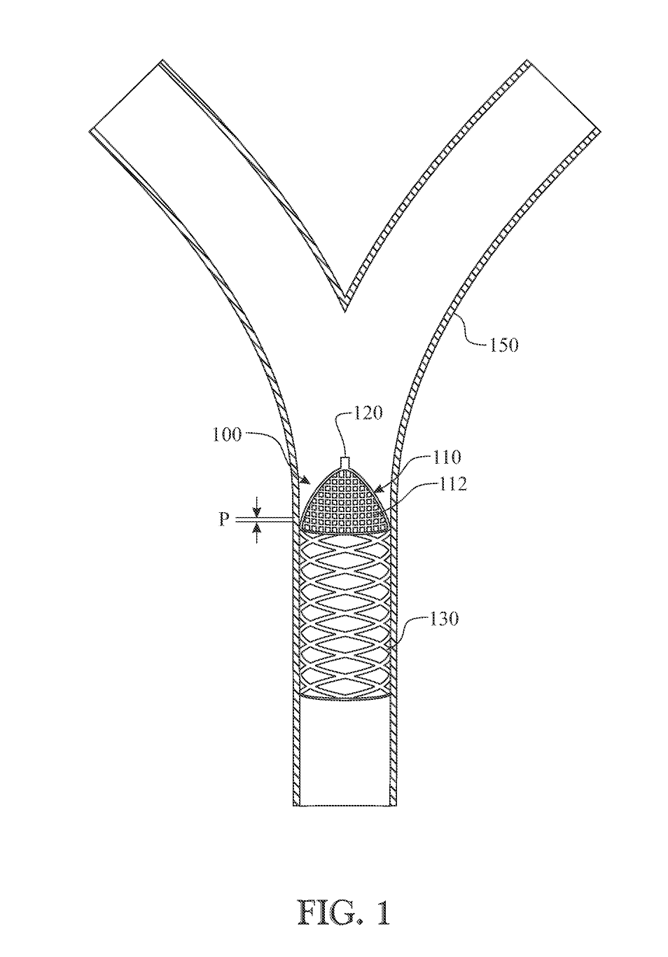

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

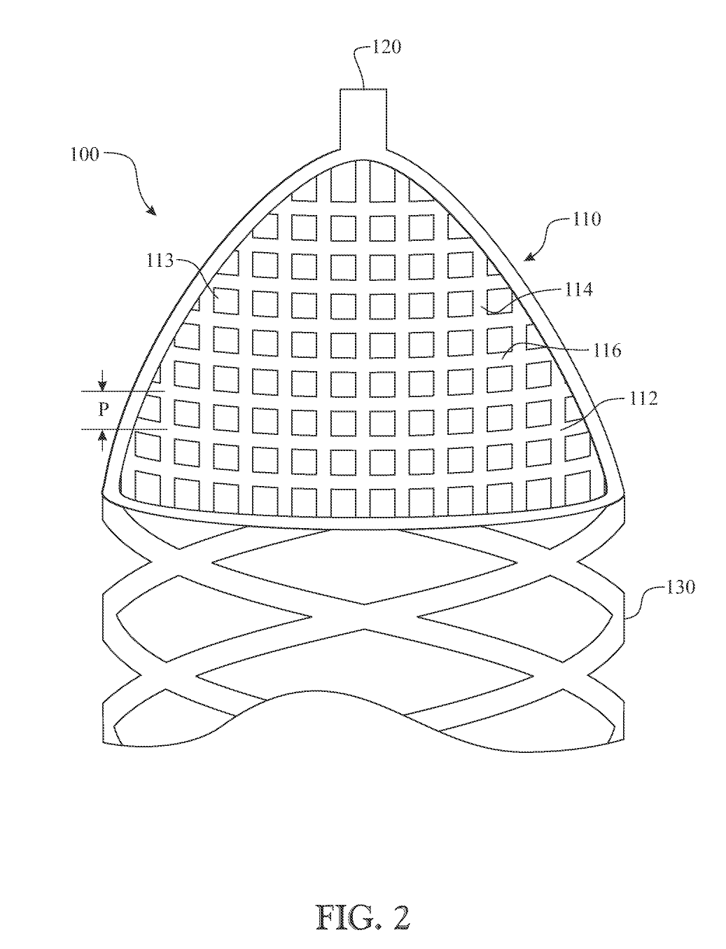

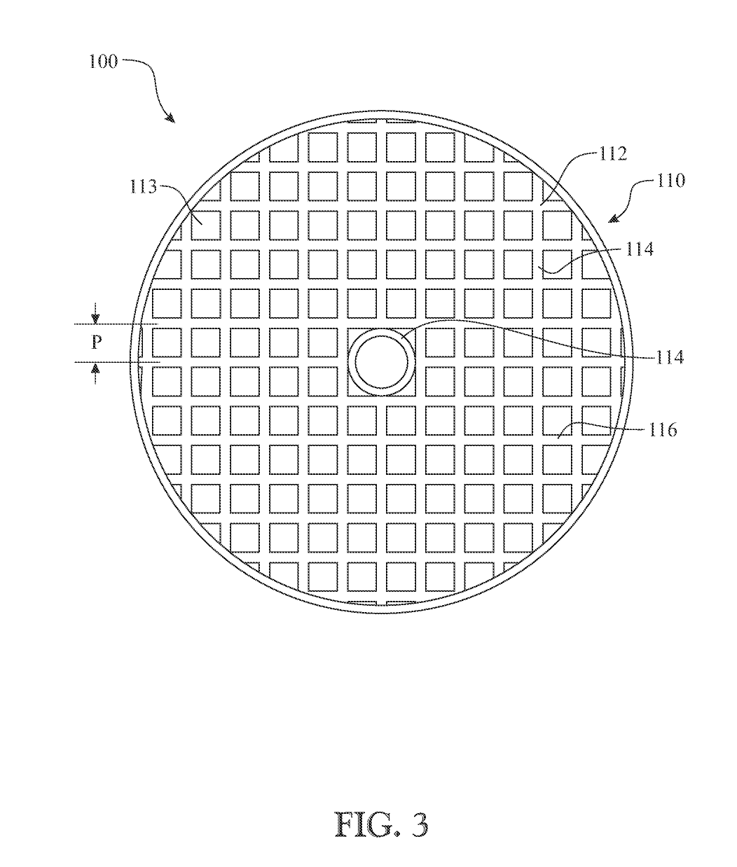

A first exemplary embodiment of an arterial emboli dissolution filter apparatus 100 is illustrated in FIGS. 1 through 3. The arterial emboli dissolution filter apparatus 100 is surgically implanted into a vessel wall 150 of a patient using common stent implanting techniques. The arterial emboli dissolution filter apparatus 100 is implanted in a target vessel 150 in a region free of plaque. The preferred implant locations will be the common carotid artery on the left and either the brachiocephalic or common carotid artery on the right. With placement in these locations, the device can retain any potential embolic debris and thus prevent an embolic stroke. The flowing blood will eventually cause lysis and subsequent resolution of the thrombus. In some circumstances, imaging studies might reveal a trapped embolism that is not dissolving. In those cases, it may be necessary to perform a procedure whereby the target blood vessel is temporarily occluded and the retained thrombus is removed. A flow sensor or other sensing mechanism may be included to signal that an embolism has been retained which is not dissolving, thus blocking blood flow. The preferred embodiment details a conical shape to the filter mesh although alternative form factors such as the filter mesh oriented perpendicular to the target vessel lumen can be utilized.

The arterial emboli dissolution filter apparatus 100 is fabricated combining a dissolution filter 110 and a tubular support section 130. The dissolution filter 110 is fabricated having a filter mesh 112. The filter mesh 112 can be of any reasonable mesh design. The exemplary dissolution filter 110 includes a series of uniformly spaced spatially arranged longitudinal elements 114 and a series of uniformly spaced spatially arranged lateral elements 116 forming a plurality of mesh pores 113. The spacing between adjacent fibers or mesh elements is referred to as a pitch “P”. At least a portion of the fibers creating the dissolution filter 110 can be designed to aid in dissolution of the emboli. The dissolution filter 110 is preferably provided in a conical shape, whereby the shape directs emboli and blood flow towards a central region. The unimpeded blood flow is then concentrated towards the emboli to aid in dissolving the emboli. The blood flow is directed towards the central region of the dissolution filter 110 by the formation of the filter mesh 112. The interior surfaces of the filter mesh 112 can be planar in regions proximate the outer circumference of the dissolution filter 110. The planar surface would aid in directing the blood flow towards the central region of the dissolution filter 110. The blood flow exits the filter mesh 112 by passing through the plurality of mesh pores 113. Additionally, the interior surface can include ridges and other features to aid in the breaking apart of the emboli. The tubular support section 130 is secured against an interior wall surface of the vessel wall 150, maintaining the dissolution filter 110 in position. An optional insertion guide 120 can be included in the dissolution filter 110 to aid in the implanting process. The dissolution filter 110 is designed to collapse when pulled along a longitudinal axis for aid during the implanting procedure.

Although the illustration presents aligned rectangular-shaped mesh pores 113, it is understood that the mesh pores 113 can be of any reasonable shape and pattern. Alternate embodiments can include oval shaped pores, diamond shaped pores, triangular shaped pores, hexagonal shaped pores, star shaped pores, irregularly shaped pores, pores having jagged edges, and the like. The inclusion of pointed regions can further aid in the dissolution of the emboli.

The filter mesh 112 can be fabricated using any reasonable known manufacturing technique. This includes molding, machining, laser or water jet cutting, casting, welding, plastic welding, ultrasonic welding, heat staking, tying, weaving, and the like. The mesh 112 can be fabricated of a single material formed into the desired shape; of a series of strands or other similar elements woven or bonded together to form the desired shape; and the like. A support frame member can be integrated to aid in supporting the form factor of the dissolution filter 110. A similar fabrication means can be utilized to fabricate the tubular support section 130 as well as joining the two sections together.

The arterial emboli dissolution filter apparatus 100 provides a distinct advantage over the current art. The design of the arterial emboli dissolution filter apparatus 100 directs the blood flow towards the retained emboli, utilizing the blood flow to dissolve or break apart the emboli. The existing art traps and collects the emboli. The filters redirect the blood flow around the trapped material. Eventually, the existing filters require removal and replacement, which is accomplished by a surgical procedure.

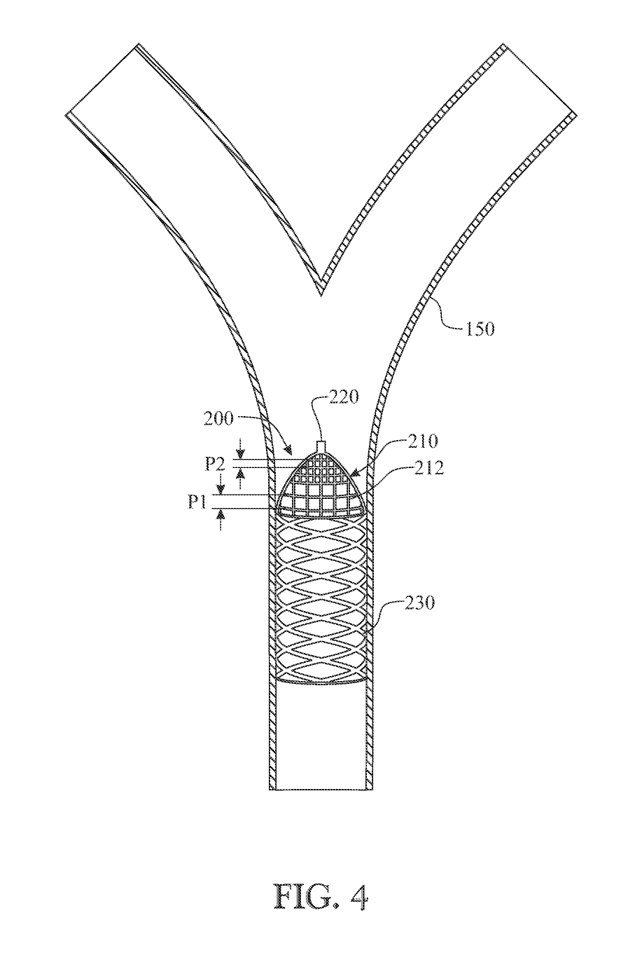

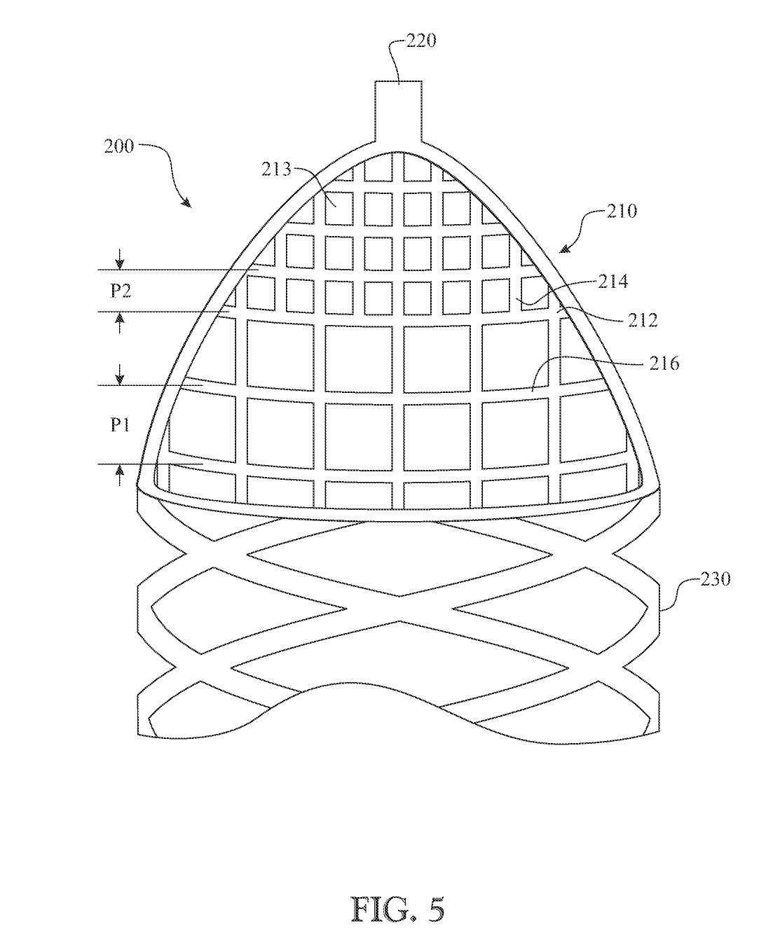

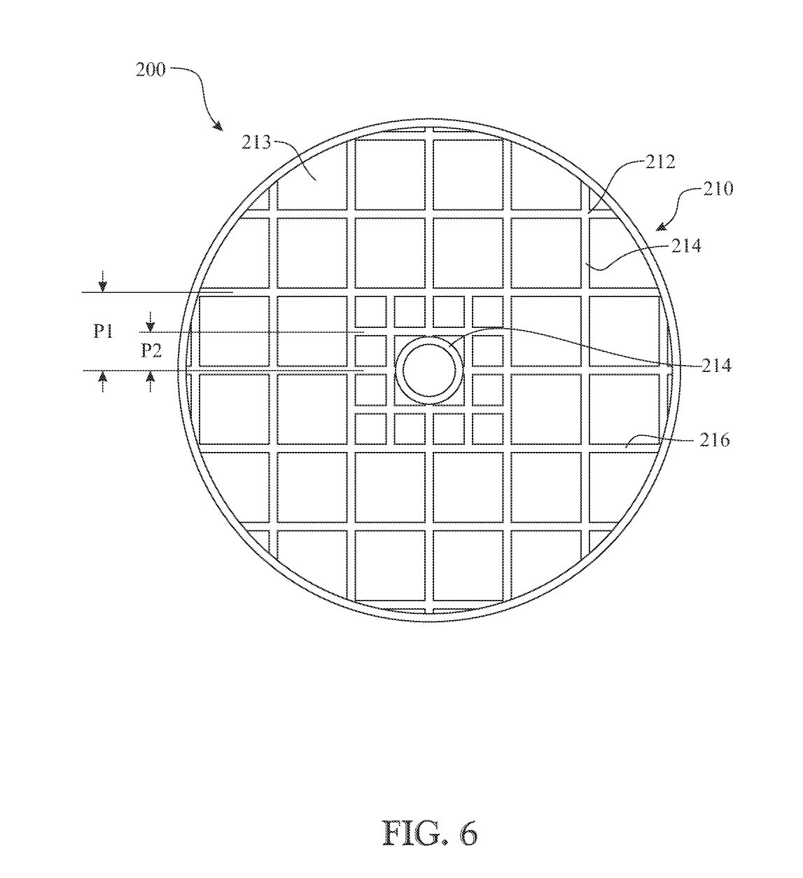

A second exemplary embodiment, referred to as an arterial emboli dissolution filter apparatus 200 is illustrated in FIGS. 4 through 6. Like features of arterial emboli dissolution filter apparatus 200 and arterial emboli dissolution filter apparatus 100 are numbered the same except preceded by the numeral ‘2 ’. Similar to the arterial emboli dissolution filter apparatus 100, the arterial emboli dissolution filter apparatus 200 is implanted in a target vessel 150 in a region free of plaque. The arterial emboli dissolution filter apparatus 100 is fabricated of a mesh having a uniform pitch P throughout. Contrarily, the arterial emboli dissolution filter apparatus 200 is fabricated of a mesh having at least two different pitches, a larger pitch P1 and a smaller pitch P2. It is understood the arterial emboli dissolution filter apparatus 200 can include a filter mesh 212 having a varied pitch throughout, preferably being sequentially reduced. The widest pitch provided proximate the tubular support section 230 interface and the tightest pitch being provided proximate an apex of the conical shape. The form factor represented by the exemplary embodiment arterial emboli dissolution filter apparatus 200 can include deflecting elements about the periphery of the filter mesh 112. These deflecting elements direct a potential embolism to the central mesh element, identified by the smaller pores 213. The purpose of the deflecting elements is that they allow for even greater flow of normal blood at the periphery of the target vessel and the central mesh only occupies a relatively small proportion of the overall vessel diameter.

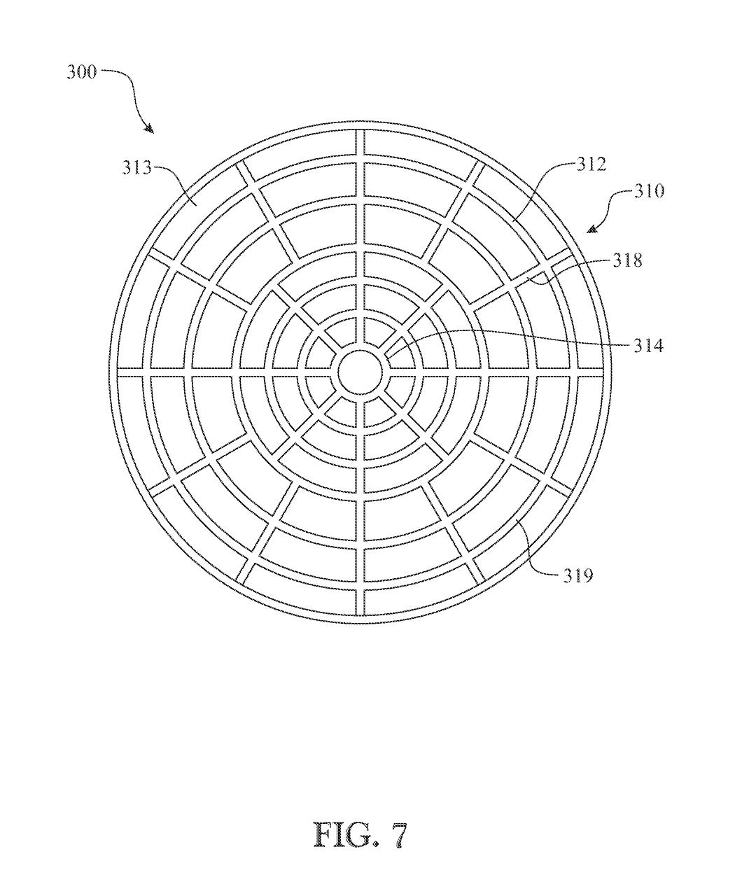

A third exemplary embodiment, referred to as an arterial emboli dissolution filter apparatus 300 is illustrated in FIG. 7. Like features of arterial emboli dissolution filter apparatus 300 and arterial emboli dissolution filter apparatus 100 are numbered the same except preceded by the numeral ‘3 ’. Similar to the arterial emboli dissolution filter apparatus 100, the arterial emboli dissolution filter apparatus 300 is implanted in a target vessel 150 in a region free of plaque. The arterial emboli dissolution filter apparatus 100, 200 are fabricated of a mesh having longitudinal and a lateral arrangement. Contrarily, the arterial emboli dissolution filter apparatus 300 is fabricated of a mesh having a plurality of angularly related spatially arranged radial elements 318 and a plurality of spatially arranged radial elements 318 of varying diameters. The passage area of pores 313 of the arterial emboli dissolution filter apparatus 300 are progressively smaller between the pores 313 along the outer circumference of the dissolution filter 310 and the pores 313 which are proximate a centered location of the dissolution filter 310 or proximate a insertion guide 320.

It is understood that arterial emboli dissolution filter apparatus 100 can comprise a plurality of filter meshes 112 provided in a serial relation, each filter 112 having a sequentially mesh pore 113 with a smaller passage area than the previous filter 112. This arrangement can be utilized to increase the affectivity of the dissolution process, wherein the first filter 112 would reduce the size of the largest emboli, the subsequent, smaller mesh pores 113 would continue to dissolve the smaller emboli that pass through the arterial emboli dissolution filter apparatus 100.

Many variations of the invention will occur to those skilled in the art. Some variations include a self-expanding stent like element while other variations call for a balloon expanding stent like element. Other variations may include a slightly larger central mesh hole to allow for over the wire delivery of the device while other variations will not need this feature.

Although the described embodiments utilize a conical shaped filter directing the blood flow towards the center, it is understood that the filter design can be of any shape and the flow of blood can be directed wherever the emboli is retained. The arterial emboli dissolution filter apparatus 100 is designed to allow the blood to flow continuously, directed towards the retained emboli.

Since many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A emboli retention and dissolution method comprising the steps of:
obtaining a emboli dissolution device, the emboli dissolution device comprising:
a dissolution filter comprising a filter mesh, the dissolution filter designed to retain emboli and direct blood flow towards the retained clots to dissolve the clots;
permanently implanting the emboli dissolution device within a blood vessel of a patient, the emboli dissolution device providing unimpeded blood flow; and
dissolving an emboli by retaining the emboli using the dissolution filter and directing blood flow towards the retained emboli.

2. A emboli retention and dissolution method as recited in claim 1, the method further comprising the step of:
utilizing an interior surface of the filter mesh to aid in the directing of blood flow towards the retained emboli.

3. A emboli retention and dissolution method as recited in claim 1, the method further comprising the step of:
utilizing an interior surface of the filter mesh to aid in the dissolution of the retained emboli.

4. A emboli retention and dissolution method as recited in claim 1, the method further comprising the step of:
elongated the emboli dissolution device along a longitudinal axis for aid in the implanting process.

5. A emboli retention and dissolution method as recited in claim 1, the method further comprising the step of:
monitoring the implanted emboli dissolution device for blockage.

6. A emboli retention and dissolution method comprising the steps of:
obtaining a emboli dissolution device, the emboli dissolution device comprising:
a filter support, and
a dissolution filter comprising a filter mesh, the dissolution filter designed to retain emboli and direct blood flow towards the retained clots to dissolve the clots;
permanently implanting the emboli dissolution device within a blood vessel of a patient by implanting the filter support into a blood vessel in a manner similar to the implanting of a stent, the emboli dissolution device providing unimpeded blood flow; and
dissolving an emboli by retaining the emboli using the dissolution filter and directing blood flow towards the retained emboli.

7. A emboli retention and dissolution method as recited in claim 6, the method further comprising the step of:
utilizing an interior surface of the filter mesh to aid in the directing of blood flow towards the retained emboli.

8. A emboli retention and dissolution method as recited in claim 6, the method further comprising the step of:
utilizing an interior surface of the filter mesh to aid in the dissolution of the retained emboli.

9. A emboli retention and dissolution method as recited in claim 6, the method further comprising the step of:
elongated the emboli dissolution device along a longitudinal axis for aid in the implanting process.

10. A emboli retention and dissolution method as recited in claim 6, the method further comprising the step of:
monitoring the implanted emboli dissolution device for blockage.

11. A emboli retention and dissolution method comprising the steps of:
obtaining a emboli dissolution device, the emboli dissolution device comprising:
a filter support, and
a dissolution filter comprising a filter mesh, the filter mesh having a series of elements forming a lattice having a series of mesh pores wherein the, the dissolution filter designed to retain emboli and direct blood flow towards the retained clots to dissolve the clots, the elements having a planar interior surface proximate the outer edges of the filter mesh;
managing retention of emboli and blood flow by incorporating mesh pores of various sizes;
permanently implanting the emboli dissolution device within a blood vessel of a patient, the emboli dissolution device providing unimpeded blood flow; and
dissolving an emboli by retaining the emboli using the dissolution filter and directing blood flow towards the retained emboli.

12. A emboli retention and dissolution method as recited in claim 11, the method further comprising the step of:
utilizing an interior surface of the filter mesh to aid in the directing of blood flow towards the retained emboli.

13. A emboli retention and dissolution method as recited in claim 11, the method further comprising the step of:
utilizing an interior surface of the filter mesh to aid in the dissolution of the retained emboli.

14. A emboli retention and dissolution method as recited in claim 11, the method further comprising the step of:
monitoring the implanted emboli dissolution device for blockage.
